(19) United States
(12) Patent Application Publication
(10) Pub. No.: US 2020/0359577 A1
(43) Pub. Date: Nov. 19, 2020

(54) METHOD FOR CONTROLLING SERINE CONTENT OF SOLANACEOUS PLANT, SOLANACEOUS PLANT, AND METHOD FOR IDENTIFYING SOLANACEOUS PLANT CULTIVATION METHOD

(71) Applicant: SEKISUI CHEMICAL CO., LTD., Osaka (JP)

(72) Inventors: Seigo ONO, Tsukuba-shi (JP); Hiroaki GOTOU, Tsukuba-shi (JP)

(73) Assignee: SEKISUI CHEMICAL CO., LTD., Osaka (JP)

(21) Appl. No.: 16/638,190

(22) PCT Filed: Sep. 28, 2018
(86) PCT No.: PCT/JP2018/036572

§ 371 Date: Feb. 11, 2020

(30) Foreign Application Priority Data
Sep. 29, 2017 (JP) 2017-189563
Sep. 29, 2017 (JP) 2017-189564
Sep. 29, 2017 (JP) 2017-189736

(51) Int. Cl. A01G 22/05; A01G 31/00; A01G 7/00

(52) U.S. Cl. CPC A01G 22/05; A01G 7/00; A01G 31/00

(57) ABSTRACT

A method for controlling a serine content of a solanaceous plant, comprising performing cultivation of a solanaceous plant using a cultivation solution containing sodium chloride, while adjusting one or both of concentration of sodium chloride in the cultivation solution and number of days for performing the cultivation using the cultivation solution.

### TECHNICAL FIELD

The present invention relates to a method for controlling a serine content of a solanaceous plant, a solanaceous plant, and a method for identifying solanaceous plant cultivation method.

Priorities are claimed on Japanese Patent Application Nos. 2017-189563, 2017-189564, and 2017-189736, each filed Sep. 29, 2017, the contents of which are incorporated herein by reference.

### DESCRIPTION OF RELATED ART

In recent years, there is a growing public concern for health, and foods containing many functional components such as amino acids have been demanded. For increasing the amount of GABA which is known to have anti-stress action, Patent Document 1 discloses a method for producing a food derived from tomato having a high GABA content by fermenting a processed tomato product with lactic acid bacteria.

However, there are also many functional components other than GABA. In order to meet general public's health-oriented desire to take as many functional components including those other than GABA as possible, it is necessary to develop combination food products including such other functional components as well.

For example, it is generally known that serine has an effect of improving sleep quality, and its effectiveness is advocated in a modern society filled with stresses and the like. In addition, it is known that serine has an effect of improving brain function, and the potential effect of reducing risk of Alzheimer's disease has also been suggested (Non-Patent Document 1).

As cultivation methods for tomatoes, hydroponic cultivation is known as well as soil cultivation.

Patent Document 2 proposes a method for hydroponically cultivating a tomato with a cultivation solution containing sodium chloride. According to the method of Patent Document 2, tomatoes with high sugar content can be produced. However, it was difficult to distinguish cultivation methods from harvested tomatoes.

Methods for increasing the amino acid contents in plants have been proposed for the purpose of improving flavor and nutritional value of the plants. Patent Document 3 proposes a method of controlling amino acid contents by gene modification technology. However, there is concern about genetically modified plants; therefore, it is desired to develop a method that can control the amino acid contents without using gene modification technology.

### PRIOR ART REFERENCES

### Patent Document

Patent Document 1: Japanese Patent Granted Publication No. 4757569
Patent Document 2: Japanese Unexamined Patent Application, First Publication No. Hei 10-271924
Patent Document 3: Japanese Unexamined Patent Application Publication (Translation of PCT Application) No. 2003-509055

### Non-Patent Document

Non-Patent Document1: Metcalf et al., Neurotox Res. 2018, 33(1), p. 213-221.

### SUMMARY OF THE INVENTION

### Problems to be Solved by the Invention

In view of the problems described above, an object of the present invention is to provide a method for controlling a serine content of a solanaceous plant.

Another object of the present invention is to provide a solanaceous plant which contains large amounts of amino acids, preferably serine, as functional components.

Still another object of the present invention is to provide a method for identifying a solanaceous plant cultivation method.

### Means to Solve the Problems

Specifically, the present invention has the following embodiments.

[1] A method for controlling a serine content of a solanaceous plant, comprising performing cultivation of a solanaceous plant using a cultivation solution containing sodium chloride, while adjusting one or both of concentration of sodium chloride in the cultivation solution and number of days for performing the cultivation using the cultivation solution.
[2] The method according to [1], wherein the cultivation is hydroponic cultivation.
[3] The method according to [1] or [2], wherein the concentration of sodium chloride in the cultivation solution is 1% by mass or more.
[4] The method according to any one of [1] to [3], wherein the number of days for performing the cultivation using the cultivation solution is ½ or more of the entire cultivation period.
[5] The method according to any one of [1] to [4], wherein the solanaceous plant is a tomato.
[6] A solanaceous plant having a serine molar content of 8 mol % or more per 100 mol % of total amino acids content of the solanaceous plant.
[7] The solanaceous plant according to [6], which has a total amino acid content of 300 mg or more per 100 g of an edible portion of the solanaceous plant.
[8] The solanaceous plant according to claim [5] or [7], which has a serine mass content of 30 mg or more per 100 g of the edible portion.
[9] The solanaceous plant according to any one of [6] to [8], which is a tomato.
[10] A cultivation method identifying method for degerming whether a solanaceous plant is one produced by hydroponic cultivation with a cultivation solution containing 1% by mass or more of sodium chloride, comprising:

measuring amino acid contents in terms of amounts (mg) of respective amino acids present per 100 g of an edible portion of a solanaceous plant,

calculating a total amino acids content α (mg) per 100 g of the edible portion of the solanaceous plant from the amino acid contents,

calculating a serine content β (mol %) relative to 100 mol % of total amino acids content, and

determining that the solanaceous plant is one produced by hydroponics with a cultivation solution containing 1% by mass or more of sodium chloride when the total amino acid content α is not less than a predetermined value α₀ and the serine content β is not less than a predetermined value β_{S}.

[11] The method according to [10], wherein the predetermined value α0 is 300 mg, and the predetermined value Ps is 8 mol %.
[12] The method according to [10] or [11], wherein the solanaceous plant is a tomato.

### Effects of Invention

The present invention can provide a method for controlling a serine content of a solanaceous plant.

The present invention can also provide a solanaceous plant with higher concentrations of amino acids as functional components, as compared to conventional solanaceous plants. Further, the present invention can provide a solanaceous plant with higher serine content among all amino acids, as compared to conventional solanaceous plants. Thus, in particular, the present invention can provide a solanaceous plant which enables efficient ingestion of serine.

The present invention can also provide a method for identifying solanaceous plant cultivation method.

### BRIEF DESCRIPTION OF DRAWINGS

FIG. 1 is a graph showing the relationship between sodium chloride content and serine content (mg/100 g of edible portion).

FIG. 2 is a graph showing the relationship between sodium chloride content and serine content (mol %/100 mol % of total amino acids).

### DESCRIPTION OF THE EMBODIMENTS

### <<Method for Controlling Serine Content of Solanaceous Plant>>

The method of the present invention for controlling a serine content of a solanaceous plant includes performing cultivation of a solanaceous plant using a cultivation solution containing sodium chloride, while adjusting concentration of sodium chloride in the cultivation solution or number of days for performing the cultivation using the cultivation solution.

The method of the present invention for controlling a serine content of a tomato may include adjusting both of the concentration of sodium chloride in the cultivation solution and the number of days for performing the cultivation using the cultivation solution.

Solanaceae is one of the families of the order Solanales, subclass Asteridae, class Magnoliopsida, and is a large group including about 90 genera. In the present invention, the solanaceous plant is preferably an edible plant. Examples of the solanaceous plant include plants of the genus Solanum, such as an eggplant (Solanum melongena), a potato (Solanum tuberosum), a pepino (Solanum muricatum Aiton), and a tomato (Solanum lycopersicum); plants of the genus Capsicum, such as a capsicum (green pepper, paprika) (Capsicum annuum), a rocoto (Capsicum pubescens), an ulupica (Capsicum cardenasii), an ají amarillo (Capsicum baccatum), a habanero (Capsicum chinense), and a bird pepper (Capsicum frutescens); plants of the genus Physalis, such as a ground cherry (Physalis alkekengi var. franchetii), and a tomatillo (Physalis ixocarpa); and plants of the genus Lycium, such as Chinese wolfberry (Lycium rhombifolium). Of these, a tomato is preferable.

The cultivation is preferably performed by hydroponic cultivation. The hydroponic cultivation is advantageous, for example, in that, since it is easy to control the concentration of sodium chloride in the cultivation solution, it is easy to adjust the amount of sodium chloride to be absorbed by the solanaceous plants, and the risk of infection is low since the kinds and amounts of pathogenic bacteria and viruses present in the cultivation solution are small as compared to the case of soil. Particularly, it is preferable to perform the cultivation according to the solanaceous plant production method to be described later.

With respect to the lower limit of the sodium chloride concentration of the cultivation solution, the sodium chloride concentration is preferably 1% by mass or more, more preferably 1.5% by mass or more, further preferably 2.0% by mass or more, and particularly preferably 2.5% by mass or more. With respect to the upper limit, the concentration is preferably 4.0% by mass or less, more preferably 3.5% by mass or less, and further preferably 3.0% by mass or less. The sodium chloride concentration of the cultivation solution is preferably in the range of 1 to 4.0% by mass or more, more preferably 1 to 3.5% by mass, further preferably 1 to 3.0% by mass, and particularly preferably 2 to 3.0% by mass. When the concentration of sodium chloride in the cultivation solution is within the above range, it is possible to increase the amount and concentration of amino acid components in the solanaceous plant and to provide foods with improved flavor and health function. In addition, the salt concentration in the solanaceous plant can be increased, so that an effect of improving shelf life can be obtained.

With respect to the number of days for performing the cultivation using the cultivation solution, the more the number of days for the cultivation, the more preferable. The lower limit of the number of days for the cultivation is preferably ⅓ or more, more preferably ½ or more, still more preferably ⅔ or more, still more preferably 9/13 or more, and even more preferably 10/13 or more, relative to the entire cultivation period. The cultivation using the cultivation solution may be terminated before harvesting, and the cultivation may be switched to a cultivation that does not use the cultivating solution, or uses a solution devoid of some of the components of the cultivation solution (for example, a solution lacking sodium chloride) or a dilution of the cultivation solution (for example, a nutrient solution having a lower sodium chloride concentration than the cultivating solution), but it is most preferable to perform the cultivation until harvesting.

The upper limit of the number of days for performing the cultivation using the cultivation solution is not particularly limited, and may be preferably ⅞ or less, more preferably 9/10 or less, further preferably 12/13 or less, relative to the entire cultivation period. However, the upper limit is most preferably the entire cultivation period (1/1). That is, the range of the number of days for performing the cultivation using the cultivation solution is preferably 1/3 to 1/1, more preferably 1/2 to 1/1, more preferably 2/3 to 1/1, more preferably 9/13 to 1/1, still more preferably 10/13 to 1/1, relative to the entire cultivation period.

When the number of days for cultivation is not less than the above-mentioned lower limit, it is possible to increase the amount and concentration of amino acid components in the solanaceous plant and to provide foods with improved flavor and health function. In addition, the salt concentration in the solanaceous plant can be increased, so that an effect of improving shelf life can be obtained.

The “entire cultivation period” means a period (number of days) of from germination until harvesting of the fruit of the solanaceous plant. However, when the “salt tolerance imparting treatment” to be described later is performed, the entire cultivation period is a period (number of days) of from immediately after initiation of contacting the plant with the salt tolerance imparting agent until harvesting of the fruit of the solanaceous plant.

When the solanaceous plant is a tomato, the degree of ripening of tomato fruit can be classified by the extent (area %) of red or pink coloration on the fruit surface. For example, a tomato experiences a green ripe period (no coloration), a degreening period (up to 70% coloration), a mature period (71 to 90% coloration), and a ripe period (91 to 100% coloration) to reach an over-ripe period. In the present invention, it is preferable to control the serine content of an edible part of a tomato at the mature stage or ripe stage.

In the present specification, the “edible part” means a part of the harvested fruits of tomato or the like excluding calyx and stem. The fruits are non-processed fruits. However, in the case of potatoes, the edible part is a tuber.

In the present specification, “amino acids” refer to free amino acids.

The amounts of amino acids can be measured by a known method. For example, a commercially available amino acid automatic analyzer can be used, and the amounts of amino acids can be determined by an amino acid automatic analysis method or a high performance liquid chromatography.

The solanaceous plant according to the present invention is preferably not a genetically modified plant.

In the present invention, the serine content of a solanaceous plant can be controlled by adjusting concentration of sodium chloride in the cultivation solution or number of days for performing the cultivation using the cultivation solution.

First, solanaceous plants are cultivated under multiple different cultivation conditions in which the concentration of sodium chloride in the cultivation solution or the number of days for performing the cultivation is varied.

Subsequently, the amino acid contents in terms of amounts of respective amino acids present per 100 g of an edible portion of the solanaceous plants cultivated under the cultivation conditions described above are measured. The “amino acid contents” denote the respective amounts of serine, glutamic acid, aspartic acid, arginine, isoleucine, alanine, lysine, histidine, phenylalanine, tyrosine, leucine, methionine, valine, glycine, proline, threonine, tryptophan, and cystine.

The amino acid contents are converted into molar amounts and added up to calculate the total amino acids content (mol). Further, the serine molar content (mol %) per 100 mol % of total amino acids content is calculated. The values of the serine molar content and the sodium chloride concentration of the cultivation solution are respectively plotted on the horizontal axis and the vertical axis to create a calibration curve (1). The values of the serine content and the cultivation days are respectively plotted on the horizontal axis and the vertical axis to create a calibration curve (2).

From the calibration curve (1), it is possible to estimate a relationship between the sodium chloride concentration of the cultivation solution and the resulting serine content.

From the calibration curve (2), it is possible to estimate a relationship between the cultivation days and the resulting serine content.

Therefore, the serine content of a solanaceous plant can be controlled to a desired level by adjusting concentration of sodium chloride in the cultivation solution or number of days for performing the cultivation using the cultivation solution.

The exposure of a solanaceous plant to sodium chloride can be determined by formula (1) below.

Exposure to sodium chloride=[concentration of sodium chloride in cultivation solution (% by mass)]×[days of cultivation using cultivation solution(days)] (1)

By adjusting the exposure to sodium chloride, the serine content of a solanaceous plant can be controlled.

First, solanaceous plants are cultivated under multiple different cultivation conditions in which the concentration of sodium chloride in the cultivation solution or the number of days for performing the cultivation is varied.

Subsequently, the amino acid contents in terms of amounts of respective amino acids present per 100 g of an edible portion of the solanaceous plants cultivated under the cultivation conditions described above are measured.

The amino acid contents are converted into molar amounts and added up to calculate the total amino acids content (mol). Further, the serine molar content (mol %) per 100 mol % of total amino acids content is calculated. The values of the serine content and the exposure to sodium chloride are respectively plotted on the horizontal axis and the vertical axis to create a calibration curve (3).

From the calibration curve (3), it is possible to estimate a relationship between the exposure to sodium chloride and the resulting serine content. Therefore, by adjusting the exposure to sodium chloride such that a desired serine content of a solanaceous plant can be achieved, the serine content of a solanaceous plant can be controlled.

With respect to the lower limit of the total amount of amino acids (total amino acids content) contained in the edible portion of a solanaceous plant per 100 g of the edible portion thereof, it is preferable to control the total amount of amino acids to 300 mg or more, more preferably 350 mg or more, still more preferably 400 mg or more, still more preferably 450 mg or more, still more preferably 500 mg or more, still more preferably 750 mg or more, and even more preferably 1000 mg or more. With respect to the upper limit of the total amino acids content, the total amount is preferably as large as possible. For example, it is preferable to control the total amount to 2000 mg or less, more preferably 1500 mg or less, still more preferably 1250 mg or less, still more preferably 1200 mg or less, still more preferably 1150 mg or less, still more preferably 1100 mg or less, still more preferably 1050 mg or less. Specifically, the total amino acids content per 100 g of the edible portion of the solanaceous plant is preferably in the range of 300 to 2000 mg, more preferably 350 to 1500 mg, still more preferably 400 to 1250 mg, and particularly preferably 450 to 1200 mg. In the present specification, the total amino acids content means a total of the amounts of serine, glutamic acid, aspartic acid, arginine, isoleucine, alanine, lysine, histidine, phenylalanine, tyrosine, leucine, methionine, valine, glycine, proline, threonine, tryptophan, and cystine.

Serine has effects of improving sleep quality and brain function assistance. In the present invention, it is preferred to control the serine molar content to 8 mol % or more, relative to 100 mol % of the total amino acids content. By controlling the serine content, a solanaceous plant having a desired serine content can be produced.

### <<Solanaceous Plant>>

The solanaceous plant of the present invention has a serine content of 8 mol % or more, relative to 100 mol % of total amino acids content of the solanaceous plant. The kind of solanaceous plant is as described above.

Regarding the value of the amino acid contents, when the solanaceous plant is a tomato, it is preferable that the edible part of the tomato at the mature stage or ripe stage satisfies the above-mentioned requirement. The mature stage and ripe stage are as described above.

In the present specification, “amino acids” refer to free amino acids. Specifically, the amino acids include serine, glutamic acid, aspartic acid, arginine, isoleucine, alanine, lysine, histidine, phenylalanine, tyrosine, leucine, methionine, valine, glycine, proline, threonine, tryptophan, and cystine.

The amounts of amino acids can be measured by a known method as mentioned above.

When the solanaceous plant of the present invention is a tomato (Solanum lycopersicum), the specific type and variety thereof is not particularly limited, but may be, for example, large-fruited tomatoes, medium-fruited tomatoes, small-fruited tomatoes, Italian tomatoes, black tomatoes, green tomatoes, fruit tomatoes and the like.

Specific examples of the large-fruited tomatoes include Momotaro and First Tomato. Specific examples of the medium-fruited tomatoes include Frutica and Red Ole. Specific examples of the small-fruited tomatoes include Aiko, Chica and micro-tomato. In the present invention, the tomato may be a genetically modified plant; however, being for food, the genetically modified tomato may not be preferable depending on the country or the legal regulation.

With respect to the lower limit of the sodium content of the solanaceous plant (for example, a fruit of a tomato) as measured with respect to an edible portion thereof, the sodium content is preferably 0.10% by mass or more, and more preferably 0.15% by mass or more. With respect to the upper limit, the sodium content is preferably 0.5% by mass or less, more preferably 0.4% by mass or less, and further preferably 0.3% by mass or less. Specifically, the sodium content of the edible portion of the solanaceous plant (e.g., a fruit of tomato) is preferably in the range of 0.10 to 0.5% by mass, more preferably 0.15 to 0.4% by mass. When the sodium content is within the above range, the flavor and shelf life of the solanaceous plant can be improved.

The present invention can provide a solanaceous plant with higher concentrations of amino acids as compared to conventional solanaceous plants. Further, the present invention can provide a solanaceous plant which has especially high concentration of serine among all enriched amino acids, as compared to conventional solanaceous plants. As described later, serine has an effect of improving sleep quality, and its effectiveness is advocated in a modern society filled with stresses and the like.

The present invention can provide a solanaceous plant enriched in specific components in the form of a natural vegetable without artificial modifications or the like. In addition, since specific amino acids are enriched, the present invention can offer advantages, for example, in that the concentration process can be reduced or omitted in the development of foods such as supplements which are enriched in specific amino acids.

Serine has effects of improving sleep quality, depression and brain function. With respect to the lower limit of the serine molar content of the solanaceous plant relative to 100 mol % of total amino acids content, the serine molar content is preferably 5.0 mol % or more, more preferably 8.0 mol % or more, more preferably 8.5 mol % or more, more preferably 8.8 mol % or more, even more preferably 9.0 mol % or more. With respect to the upper limit, the serine molar content is preferably as high as possible, and may be, for example, preferably 30.0 mol % or less, more preferably 20.0 mol % or less, more preferably 15.0 mol % or less, more preferably 14.0 mol % or less, more preferably 13.5 mol % or less, even more preferably 13.0 mol % or less. Specifically, the serine molar content of the solanaceous plant relative to 100 mol % of total amino acids content is preferably in the range of 5.0 to 30.0 mol %, more preferably 8.0 to 20.0 mol %, still more preferably 8.5 to 15.0 mol %, still more preferably 8.5 to 14.0 mol %, and particularly preferably 8.5 to 13.5 mol %.

Glutamic acid has effects of improving flavor and promoting fatigue recovery. With respect to the lower limit of the glutamic acid molar content of the solanaceous plant relative to 100 mol % of total amino acids content, the glutamic acid molar content is preferably 50.0 mol % or more, more preferably 60 mol % or more, even more preferably 62.0 mol % or more. With respect to the upper limit, the glutamic acid molar content is preferably as high as possible, and may be, for example, preferably 65.0 mol % or less, more preferably 63.0 mol % or less. Specifically, the glutamic acid molar content of the solanaceous plant relative to 100 mol % of total amino acids content is preferably in the range of 50.0 to 65.0 mol %, more preferably 50.0 to 63.0 mol %.

Aspartic acid has an effect of promoting fatigue recovery.

With respect to the lower limit of the aspartic acid molar content of the solanaceous plant relative to 100 mol % of total amino acids content, the aspartic acid molar content is preferably 9.0 mol % or more, more preferably 9.5 mol % or more, even more preferably 10.5 mol % or more. With respect to the upper limit, the aspartic acid molar content is preferably as high as possible, and may be, for example, preferably 13.0 mol % or less. Specifically, the aspartic acid molar content of the solanaceous plant relative to 100 mol % of total amino acids content is preferably in the range of 9.0 to 13.0 mol %.

Arginine has effects of improving immune function, and vasodilating action.

With respect to the lower limit of the arginine molar content of the solanaceous plant relative to 100 mol % of total amino acids content, the arginine molar content is preferably 0.5 mol % or more, more preferably 0.6 mol % or more. With respect to the upper limit, the arginine molar content is preferably as high as possible, and may be, for example, preferably 1.8 mol % or less, more preferably 1.5 mol % or less.

Specifically, the arginine molar content of the solanaceous plant relative to 100 mol % of total amino acids content is preferably in the range of 0.5 to 1.8 mol %.

Isoleucine has effects such as vasodilation, growth promotion, muscle strengthening, and fatigue recovery.

With respect to the lower limit of the isoleucine molar content of the solanaceous plant relative to 100 mol % of total amino acids content, the isoleucine molar content is preferably 0.9 mol % or more, more preferably 1.0 mol % or more, more preferably 1.2 mol % or more, even more preferably 1.5 mol % or more. With respect to the upper limit, the isoleucine molar content is preferably as high as possible, and may be, for example, preferably 2.80 mol % or less, more preferably 1.58 mol % or less. Specifically, the isoleucine molar content of the solanaceous plant relative to 100 mol % of total amino acids content is preferably in the range of 0.9 to 2.80 mol %.

Alanine has effects such as improvement of immune function and improvement of activity of liver.

With respect to the lower limit of the alanine molar content of the solanaceous plant relative to 100 mol % of total amino acids content, the alanine molar content is preferably 0.4 mol % or more, more preferably 0.5 mol % or more, more preferably 0.6 mol % or more, even more preferably 0.7 mol % or more. With respect to the upper limit, the alanine molar content is preferably as high as possible, and may be, for example, preferably 2.0 mol % or less, more preferably 1.5 mol % or less. Specifically, the alanine molar content of the solanaceous plant relative to 100 mol % of total amino acids content is preferably in the range of 0.4 to 2.0 mol %, more preferably 0.5 to 2.0 mol %.

Lysine has effects of body tissue repairing and virus inhibition.

With respect to the lower limit of the lysine molar content of the solanaceous plant relative to 100 mol % of total amino acids content, the lysine molar content is preferably 0.6 mol % or more, more preferably 0.7 mol % or more, more preferably 0.9 mol % or more, even more preferably 1.4 mol % or more. With respect to the upper limit, the lysine molar content is preferably as high as possible, and may be, for example, preferably 2.00 mol % or less, more preferably 1.9 mol % or less. Specifically, the lysine molar content of the solanaceous plant relative to 100 mol % of total amino acids content is preferably in the range of 0.6 to 2.00 mol %.

Histidine has effects of leukocyte/erythrocyte formation and appetite suppression.

With respect to the lower limit of the histidine molar content of the solanaceous plant relative to 100 mol % of total amino acids content, the histidine molar content is preferably 1.0 mol % or more, more preferably 1.2 mol % or more, more preferably 1.4 mol % or more, even more preferably 1.7 mol % or more. With respect to the upper limit, the histidine molar content is preferably as high as possible, and may be, for example, preferably 2.5 mol % or less, more preferably 2.2 mol % or less. Specifically, the histidine molar content of the solanaceous plant relative to 100 mol % of total amino acids content is preferably in the range of 1.0 to 2.5 mol %.

Phenylalanine has analgesic action, antidepressant action, and memory improvement effect.

With respect to the lower limit of the phenylalanine molar content of the solanaceous plant relative to 100 mol % of total amino acids content, the phenylalanine molar content is preferably 0.5 mol % or more, more preferably 0.75 mol % or more, more preferablyl.2 mol % or more, more preferably 1.9 mol % or more. With respect to the upper limit, the phenylalanine molar content is preferably as high as possible, and may be, for example, preferably 2.5 mol % or less, more preferably 2.0 mol % or less. Specifically, the phenylalanine molar content of the solanaceous plant relative to 100 mol % of total amino acids content is preferably in the range of 0.5 to 2.5 mol.

Tyrosine has an effect of concentration improvement.

With respect to the lower limit of the tyrosine molar content of the solanaceous plant relative to 100 mol % of total amino acids content, the tyrosine molar content is preferably 0.1 mol % or more, 0.4 mol % or more, more preferably 0.6 mol % or more. With respect to the upper limit, the tyrosine molar content is preferably as high as possible, and may be, for example, preferably 0.8 mol % or less, more preferably 0.7 mol % or less. Specifically, the tyrosine molar content of the solanaceous plant relative to 100 mol % of total amino acids content is preferably in the range of 0.1 to 0.8 mol %.

Leucine has effects such as muscle strengthening and liver function improvement.

With respect to the lower limit of the leucine molar content of the solanaceous plant relative to 100 mol % of total amino acids content, the leucine molar content is preferably 0.4 mol % or more, more preferably 0.6 mol % or more, even more preferably 0.75 mol % or more. With respect to the upper limit, the leucine molar content is preferably as high as possible, and may be, for example, preferably 1.2 mol % or less, more preferably 1.0 mol % or less, even more preferably 0.9 mol % or less. Specifically, the leucine molar content of the solanaceous plant relative to 100 mol % of total amino acids content is preferably in the range of 0.4 to 1.2 mol %, more preferably 0.4 to 1.0 mol %, more preferably 0.4 to 0.9 mol %.

Methionine has allergic reaction suppressing effect, antidepressive effect, and liver/kidney function improving effect.

With respect to the lower limit of the methionine molar content of the solanaceous plant relative to 100 mol % of total amino acids content, the methionine molar content is preferably 0.1 mol % or more, more preferably 0.2 mol % or more, even more preferably 0.3 mol % or more. With respect to the upper limit, the methionine molar content is preferably as high as possible, and may be, for example, preferably 0.4 mol % or less. Specifically, the methionine molar content of the solanaceous plant relative to 100 mol % of total amino acids content is preferably in the range of 0.1 to 0.4 mol %.

Valine has effects such as growth promotion and liver function improvement.

With respect to the lower limit of the valine molar content of the solanaceous plant relative to 100 mol % of total amino acids content, the valine molar content is preferably 0.4 mol % or more, more preferably 0.5 mol % or more, more preferably 0.8 mol %, even more preferably 1.0 mol % or more. With respect to the upper limit, the valine molar content is preferably as high as possible, and may be, for example, preferably 1.2 mol % or less, more preferably 1.0 mol % or less. Specifically, the valine molar content of the solanaceous plant relative to 100 mol % of total amino acids content is preferably in the range of 0.4 to 1.2 mol %, more preferably 0.4 to 1.0 mol %.

Glycine has an effect of improving sleep quality.

With respect to the lower limit of the glycine molar content of the solanaceous plant relative to 100 mol % of total amino acids content, the glycine molar content is preferably 0.5 mol % or more, more preferably 0.6 mol % or more, more preferably 0.7 mol %, even more preferably 0.8 mol % or more. With respect to the upper limit, the glycine molar content is preferably as high as possible, and may be, for example, preferably 1.8 mol % or less, more preferably 1.5 mol % or less. Specifically, the glycine molar content of the solanaceous plant relative to 100 mol % of total amino acids content is preferably in the range of 0.5 to 1.8 mol %.

Proline has effects of ameliorating joint pain, beautifying skin and moisturizing.

With respect to the lower limit of the proline molar content of the solanaceous plant relative to 100 mol % of total amino acids content, the valine molar content is preferably 1.0 mol % or more, more preferably 2.0 mol % or more, more preferably 5.0 mol %, even more preferably 9.0 mol % or more. With respect to the upper limit, the proline molar content is preferably as high as possible, and may be, for example, preferably 15.0 mol % or less, more preferably 11.0 mol % or less. Specifically, the proline molar content of the solanaceous plant relative to 100 mol % of total amino acids content is preferably in the range of 1.0 to 15.0 mol %, more preferably 2.0 to 11.0 mol %.

Threonine has effects such as growth promotion and liver function improvement.

With respect to the lower limit of the threonine molar content of the solanaceous plant relative to 100 mol % of total amino acids content, the threonine molar content is preferably 2.0 mol % or more, more preferably 2.2 mol % or more, even more preferably 2.5 mol % or more. With respect to the upper limit, the threonine molar content is preferably as high as possible, and may be, for example, preferably 3.0 mol % or less. Specifically, the threonine molar content of the solanaceous plant relative to 100 mol % of total amino acids content is preferably in the range of 2.0 to 3.0 mol %.

Tryptophan has tranquilizing action, analgesic action, and sleep improving effect.

With respect to the lower limit of the tryptophan molar content of the solanaceous plant relative to 100 mol % of total amino acids content, the tryptophan molar content is preferably 0.1 mol % or more, more preferably 0.2 mol % or more. With respect to the upper limit, the tryptophan molar content is preferably as high as possible, and may be, for example, preferably 0.4 mol % or less. Specifically, the tryptophan molar content of the solanaceous plant relative to 100 mol % of total amino acids content is preferably in the range of 0.1 to 0.4 mol %.

With respect to the serine mass content per 100 g of the edible portion of the solanaceous plant, the serine mass content is preferably 30 mg or more, more preferably 30 to 300 mg, still more preferably 40 to 250 mg, still more preferably 70 to 200 mg, and particularly preferably 90 to 150 mg.

With respect to the glutamic acid mass content per 100 g of the edible portion of the solanaceous plant, the glutamate mass content is preferably 200 mg or more, more preferably 200 to 2000 mg, still more preferably 300 to 800 mg, still more preferably 400 to 700 mg, particularly preferably 500 to 600 mg.

With respect to the aspartic acid mass content per 100 g of the edible portion of the solanaceous plant, the aspartic acid mass content is preferably 50 mg or more, more preferably 50 to 300 mg, more preferably 70 to 200 mg, still more preferably 85 to 180 mg, and particularly preferably 100 to 150 mg.

With respect to the arginine mass content per 100 g of the edible portion of the solanaceous plant, the arginine mass content is preferably 6 mg or more, more preferably 6 to 150 mg, more preferably 10 to 150 mg, still more preferably 15 to 100 mg, and particularly preferably 20 to 50 mg.

With respect to the isoleucine mass content per 100 g of the edible portion of the solanaceous plant, the isoleucine mass content is preferably 7 mg or more, more preferably 7 to 100 mg, still more preferably 10 to 80 mg, still more preferably 13 to 50 mg, and particularly preferably 15 to 30 mg.

With respect to the alanine mass content per 100 g of the edible portion of the solanaceous plant, the alanine mass content is preferably 8 mg or more, more preferably 8 to 200 mg, more preferably 20 to 150 mg, still more preferably 40 to 100 mg, and still more preferably 50 to 80 mg.

With respect to the lysine mass content per 100 g of the edible portion of the solanaceous plant, the lysine mass content is preferably 10 mg or more, more preferably 10 to 100 mg, still more preferably 15 to 50 mg, particularly preferably 20 to 30 mg.

With respect to the histidine mass content per 100 g of the edible portion of the solanaceous plant, the histidine mass content is preferably 10 mg or more, more preferably 10 to 100 mg, still more preferably 15 to 80 mg, particularly preferably 20 to 60 mg.

With respect to the phenylalanine mass content per 100 g of the edible portion of the solanaceous plant, the phenylalanine mass content is preferably 20 mg or more, more preferably 20 to 100 mg, still more preferably 23 to 80 mg, particularly preferably 25 to 60 mg.

With respect to the tyrosine mass content per 100 g of the edible portion of the solanaceous plant, the tyrosine mass content is preferably 7 mg or more, more preferably 7 to 100 mg, still more preferably 9 to 50 mg.

With respect to the leucine mass content per 100 g of the edible portion of the solanaceous plant, the leucine mass content is preferably 7 mg or more, more preferably 7 to 100 mg, still more preferably 7 to 50 mg.

With respect to the methionine mass content per 100 g of the edible portion of the solanaceous plant, the methionine mass content is preferably 2 mg or more, more preferably 2 to 100 mg, still more preferably 2 to 50 mg.

With respect to the valine mass content per 100 g of the edible portion of the solanaceous plant, the valine mass content is preferably 7 mg or more, more preferably 7 to 100 mg, still more preferably 7 to 50 mg.

With respect to the glycine mass content per 100 g of the edible portion of the solanaceous plant, the glycine mass content is preferably 2 mg or more, more preferably 2 to 100 mg, still more preferably 5 to 50 mg, particularly preferably 7 to 30 mg.

With respect to the proline mass content per 100 g of the edible portion of the solanaceous plant, the proline mass content is preferably 50 mg or less, more preferably 1 to 50 mg, still more preferably 5 to 40 mg, particularly preferably 10 to 30 mg.

With respect to the threonine mass content per 100 g of the edible portion of the solanaceous plant, the threonine mass content is preferably 10 mg or more, more preferably 10 to 100 mg, still more preferably 15 to 80 mg, particularly preferably 23 to 50 mg.

With respect to the tryptophan mass content per 100 g of the edible portion of the solanaceous plant, the tryptophan mass content is preferably 2 mg or more, more preferably 2 to 50 mg.

By eating a solanaceous plant containing the amino acid components in amounts as mentioned above, not only can various effects attributable to the respective amino acids be expected, but the deficiency in daily intake of the amino acids as nutrients can be compensated. In addition, the solanaceous plant can be concentrated and used as processed foods.

### <<Solanaceous Plant Production Method>>

The solanaceous plant production method of the present invention include: a salt tolerance imparting step of performing a salt resistance imparting treatment by bringing a salt tolerance imparting agent into contact with at least a part of a root of a solanaceous plant, thereby obtaining a salt tolerance imparted solanaceous plant; and a cultivation step of hydroponically cultivating the salt tolerance imparted solanaceous plant obtained in the salt tolerance imparting step with a cultivation solution having a sodium chloride concentration of 1% by mass or more. The salt tolerance imparting step is carried out for imparting salt tolerance to a solanaceous plant originally having low salt tolerance by treating the plant with the salt tolerance imparting agent, thereby enabling the plant to be cultivated under an environment of very high salinity such as the sodium chloride concentration of 1% by mass or more. In the subsequent cultivation step, the salt tolerance imparted solanaceous plant can be hydroponically cultivated with the cultivation solution having a sodium chloride concentration of 1% by mass or more.

By performing the salt tolerance imparting step and the cultivation step, it becomes possible to produce a solanaceous plant improved in terms of amino acid contents, flavor, health function and the like.

A solanaceous plant at an early stage of growth has less stress tolerance than a sufficiently grown plant, and is more likely to be influenced by environmental stress. In particular, the process of rooting or budding is very sensitive to the salinity. Therefore, when grown under a high salinity environment from the stage of seeds or bulbs, many of the solanaceous plants are caused to wither due to high salt stress before acquiring sufficient salt tolerance even if the salt tolerance imparting treatment is carried out. In the solanaceous plant production method of the present invention, it is preferable that the solanaceous plant is grown under a low salinity environment at an early stage of the growth, and the salt tolerance imparting treatment is performed after the solanaceous plant has been grown to a certain extent. This can noticeably increase a proportion of the solanaceous plants to which the salt tolerance is imparted by the salt tolerance imparting treatment, thereby making it possible to efficiently raise seedlings capable of being cultivated under a high salinity environment. As other advantages available, the salt tolerance imparting agent can be prevented from being wasted, procedure for redoing the cultivation due to unsuccessful salt tolerance imparting treatment can be reduced or omitted, and cost reduction effect and the like can be obtained.

In order to have a solanaceous plant acquire salt tolerance by the salt tolerance imparting step, this step is preferably performed after the budded seedling has been grown for at least one week, more preferably for approximately two weeks, still more preferably for approximately three weeks.

In the present invention, the salt tolerance imparting agent is used in the form of a solution thereof in water or other solvent. The solution may further include sodium chloride.

In the present invention, the salt tolerance imparting agent may be sprayed to soil or a cultivating solution for hydroponic cultivation. In order to increase the efficiency in contacting the salt tolerance imparting agent with the plant, it is preferable to allow the salt tolerance imparting agent to directly contact at least a part of the plant.

The plant may be seedlings or roots thereof. Particularly, in hydroponic cultivation, spraying the salt tolerance imparting agent directly to a cultivation solution results in unfavorable situation where the salt tolerance imparting agent is diluted and the water flow alone cannot easily provide the plant with sufficient amount of the salt tolerance imparting agent; therefore, it is preferable to allow the salt tolerance imparting agent to directly contact at least a part of the roots of the plant. The salt tolerance imparting agent may be contacted with the seedling planted on a hydroponic cultivation bed or soil, or the seedling may be planted after contacting at least a part of the roots thereof with the salt tolerance imparting agent.

The solanaceous plant production method of the present invention preferably includes, as an initial growth step, a step of allowing the plant to grow under an environment with a sodium chloride concentration of less than 1% by mass, at least until the rooting and the budding are completed. The sodium chloride concentration of the environment in which the seed or the like is grown in the initial growth step may be less than 1% by mass, and is preferably equal to or less than the salinity at which a solanaceous plant of the same variety as the solanaceous plant to be obtained by raising the seedling is capable of being normally grown. The environment that allows “capable of being normally grown” means an environment in which the cultivation of multiple solanaceous plants results in a survival rate of 80% or more. In the solanaceous plant production method of the present invention, the sodium chloride concentration of the environment for the initial growth step is preferably 0 to 0.5% by mass, more preferably 0 to 0.3% by mass, and further preferably 0 to 0.1% by mass.

The initial growth step can be performed by a general method for causing the seed or bulb to bud and root, except that an aqueous solution having a sodium chloride concentration of less than 1% by mass is used as water (initial growth solution) to be supplied to the seed or bulb. Specifically, the seed or the bulb is allowed to bud and root by placing the seed or the bulb in a state of being in contact with the initial growth solution under a temperature environment which allows budding and rooting. For example, the initial growth solution may be periodically sprayed onto the seed or the like which is placed under a suitable temperature environment. Alternatively, the seed or the like may be placed in a state where at least a part of the surface of the seed or the like is exposed to air while other parts are in contact with the initial growth solution under a suitable temperature environment. For example, the seed or the like may be placed on the surface of a supporting carrier including the initial growth solution, thereby allowing the seed or the like to be in contact partially with the initial growth solution. Alternatively, the seed or the like is placed in the initial growth solution that is held in a container such that the surface of the solution is lower than the top of the seed or the like, to thereby allow the seed or the like to be in contact partially with the initial growth solution.

The supporting carrier is not limited as long as the supporting carrier has such a porosity that allows the initial growth solution contained therein to be supplied to the seed or the like placed on the surface of the carrier. However, it is preferable that the supporting carrier has such a porosity that, after rooting, allows the root of the seedling to penetrate through the supporting carrier. By allowing the solanaceous plant budded and rooted from the seed or the like to grow such that a stem or a leaf grows upward from the supporting carrier, and the root grows downward into the supporting carrier, the plant can grow in a state of being supported by the supporting carrier. For example, when the solanaceous plant is grown such that the seed or the like is allowed to bud and root by placing the seed or the like on the surface of the supporting carrier retained within a cultivation pot installable in a cultivation tank used for the hydroponic cultivation performed in the cultivation step, while allowing the root to grow downward into the supporting carrier so as to penetrate through the supporting carrier, it is possible, even after the seedling stage, to grow the solanaceous plant by installing the cultivation pot as such in the cultivation tank, since the plant is supported in a state of being retained in the cultivation pot.

As a supporting carrier having such porosity, for example, a gel material, a fibrous material, or a granular or gravel-shaped material may be used. Examples of the gel material include polysaccharide polymers such as agar, agarose, gellan gum, and alginic acid; and water absorptive resins such as acrylic resin. Examples of the fibrous material include non-woven fabric, cotton, paper, rock wool, and glass wool. Examples of the granular or gravel-shaped material include a wood chip, a bark, pumice, vermiculite, and sand.

After the initial growth step, the salt tolerance imparting treatment may be performed, as a salt tolerance imparting step, by bringing the salt tolerance imparting agent into contact with at least a part of a root of the grown seedling. The salt tolerance imparting treatment may be performed immediately after budding and rooting, but this treatment can enhance the tolerance against salt stress more as the seedling grows more. Therefore, it is preferable that the salt tolerance imparting step is performed after the seedling is grown for at least one week, and preferably for approximately three weeks, after budding.

The salt tolerance imparting treatment can be performed by soaking at least a part of the root of the seedling into an aqueous solution (treatment solution) containing the salt tolerance imparting agent. The sodium chloride concentration of the treatment solution is not particularly limited, and may be appropriately adjusted depending on a kind of the salt tolerance imparting agent used or a kind of the solanaceous plant such that a sufficient salt tolerance imparting efficiency is obtained. For example, the treatment solution may be a solution obtained by mixing the salt tolerance imparting agent with the initial growth solution, a solution obtained by mixing the salt tolerance imparting agent with the cultivation solution used in the cultivation step, or a solution having a salt composition different from those of the initial growth solution and the cultivation solution. The sodium chloride concentration of the treatment solution used in the present invention is preferably 1% by mass or more, and more preferably the same as the sodium chloride concentration of the cultivation solution.

The salt tolerance imparting agent used in the present invention may be at least one type of drug, a microorganism, a culture supernatant of the microorganism, or combinations of these. Examples of the drug include pyrroloquinoline quinone (see Japanese Patent No. 5013326), histone deacetylase inhibitor compounds (see Japanese Unexamined Patent Application Publication No. No. 2016-69380) and strigolactone. Specific examples of the microorganisms include those belonging to the genera Azospirillum, Pseudomonas, Enterobacter, Achromobacter, Eromonas, Bacillus, Variovorax, Burkholderia, Serratia, Mycobacterium, and Paenibacillus. More specific examples of the microorganisms include those described in, for example, Plant, Cell and Environment, (2009) 32, 1682-1694 and Japanese Unexamined Patent Application, First Publication No. 2013-75881. Specific examples of the microorganisms include Azospirillum brasilense, Pseudomonas syringae, Pseudomonas fluorescens, Enterobacter aerogenes, Azospirillum, Achromobacter piechaudii, Aeromonas hydrophila/caviae, Bacillus insolitus, Variovorax paradoxus, Enterobacter cloacae, Pseudomonas putida, Burkholderia phytofirmans, Aeromonas hydrophila, Serratia liquefaciens, Serratia proteamaculans, Bacillus polymyxa, Mycobacterium phlei, Pseudomonas alcaligenes, Bacillus subtilis, Bacillus megaterium, and Paenibacillus fukuinensis. Among these, Azospirillum brasilense, Pseudomonas syringae, Pseudomonas fluorescens, Enterobacter aerogenes, Azospirillum, Achromobacter piechaudii, Aeromonas hydrophila/caviae, Bacillus insolitus and Paenibacillus fukuinensis are preferable, and Achromobacter piechaudii and Paenibacillus fukuinensis are more preferable.

The salt tolerance imparting agent used in the present invention may be Pseudomonas corrugata PP001-SRDAAT strain having 16S rDNA with a base sequence represented by SEQ ID NO: 1. The Pseudomonas corrugata PP001-SRDAAT strain was internationally deposited on Sep. 26, 2018 at the National Institute of Advanced Industrial Science and Technology under the Patent Microorganism Depositary Center (NPMD) (Room 120, Kazusa Kamisoku, Kisarazu City, Chiba Prefecture, Japan) with the deposit receipt number NITE AP-02788. The PP001-SRDAAT strain is a novel strain of Pseudomonas corrugata with a sequence identity of 16S rDNA of 99% with Pseudomonas corrugata strain E60 16S ribosomal RNA gene, partial sequence (accession number: HQ 407237.1).

The concentration of the salt tolerance imparting agent in the treatment solution can be appropriately adjusted in consideration of the kind of the salt tolerance imparting agent, the kind of the solanaceous plant, the salinity of the cultivation solution, the growth stage or the like. When the concentration of the salt tolerance imparting agent in the treatment solution is too low, the salt tolerance imparting agent is less likely to get in contact with the root of the solanaceous plant in the treatment solution, which may result in insufficient salt tolerance imparting effect. On the other hand, depending on the kind of the salt tolerance imparting agent, the growth of the solanaceous plant may be adversely affected by excessive intake of the salt tolerance imparting agent. In view of this, an appropriate concentration of the salt tolerance imparting agent in the treatment solution for obtaining sufficient salt tolerance imparting effect can be determined empirically. For example, when the salt tolerance imparting agent is the microorganism, the concentration of the microorganism in the treatment solution may be set to be 103 CFU/mL or more, whereby sufficient salt tolerance imparting effect can be obtained. When the salt tolerance imparting agent is the microorganism, the concentration of the microorganism in the treatment solution is preferably 104 CFU/mL or more, and more preferably 105 CFU/mL or more. When the salt tolerance imparting agent is the microorganism, an upper limit of the concentration of the microorganism in the treatment solution is not particularly limited, but for example, the concentration may be up to 10¹³ CFU/mL, whereby the quality of the treatment solution can be favorably maintained. The upper limit of the concentration of the microorganism in the treatment solution is preferably 10¹² CFU/mL, more preferably 10¹¹ CFU/mL, still more preferably 10¹⁰ CFU/mL, particularly preferably 10⁹ CFU/mL. The range of the concentration of the microorganism in the treatment solution may be, for example, 10³ to 10¹³ CFU/mL·^{preferably 104} to 10¹² CFU/mL, more preferably 10⁵ to 10¹¹ CFU/mL, still more preferably 10⁵ to 10¹⁰ CFU/mL, especially preferably 10⁵ to 10⁹ CFU/mL.

When the salt tolerance imparting agent is the microorganism, existing knowledge on various conditions can be relied upon regarding the culture of the microorganism, the method for separation and recovery of the microorganism, and the like.

In the present invention, the salt tolerance imparting treatment may be implemented by way of improving salt tolerance of a plant includes suppressing or inhibiting function of PERK13 (Proline-rich extensin-like receptor kinase 13) in a solanaceous plant. PERK13 is a protein involved in regulating the influx of sodium ions into plants.

Though the present invention is concerned with a solanaceous plant, the plant with its salt tolerance to be improved is not particularly limited as long as the salt tolerance imparting treatment suppresses or inhibits the function of PERK13 and the plant inherently has the PERK13 gene or a homologue gene thereof in the genomic DNA. The plant may be either an angiosperms or a gymnosperm, and may even be a fern or a moss. Further, the plant may be a monocotyledonous plant or a dicotyledonous plant. Specific examples of the plant include plants of the Gramineae family such as rice, corn, sorghum, wheat, barley, rye, barnyard millet, or foxtail millet; the plant of the Solanaceae such as tomato, eggplant, paprika, bell pepper, potato, or tobacco; plants of the Brassicaceae family such as Arabidopsis thaliana, colza, shepherd's purse, radish, cabbage, violet cabbage, Brussels sprouts (Petit vert), Chinese cabbage, bok choy, kale, watercress, Japanese mustard spinach, broccoli, cauliflower, turnip, Japanese horseradish, or mustard; plants of the Cucurbitaceae family such as cucumber, bitter melon, pumpkin, melon, or watermelon; plants of the Vitaceae family such as grape; plants of the Rutaceae family such as lemon, orange, navel orange, grapefruit, mandarin orange, lime, Citrus sudachi, citron, Shikuwasa, or citrus tankan; plants of the Rosaceae family such as apple, cherry, Japanese apricot, peach, loquat, apricot, plum (Prumus salicina), prune, almond, pear, European pear, strawberry, raspberry, blackberry, black currant, cranberry, or blueberry; plants of the Leguminosae family such as soybean, kidney bean, pea, broad bean, green soybean, green gram, or chick pea; plants of the Nelumbonaceae family such as lotus (lotus root); plants of the Pedaliaceae family such as sesame; plants of the Chenopodiaceae family such as spinach, beet, sugar beet, quinoa, tumbleweed, amaranth, or cockscomb; plants of the Arecaceae family such as date palm, oil palm, coconut, or acai; plants of the Musaceae family such as banana, Musa basjoo, or Manila hemp; plants of the Malvaceae family such as cotton or okra; plants of the Myrtaceae family such as eucalyptus; and plants of the Capparaceae family such as Cleome gynandra or Cleome spinose.

The salt tolerance of the solanaceous plants may be imparted through other known salt tolerance imparting mechanism in addition to the PERK 13.

The time period for performing the salt tolerance imparting treatment time once, that is, the time for holding at least a part of the root of the solanaceous plant to be soaked in the treatment solution, can be appropriately adjusted in consideration of the kind of the solanaceous plant or the kind of the salt tolerance imparting agent to be used. For example, such time period for the salt tolerance imparting treatment is preferably 1 hour or more, more preferably 3 hours or more, more preferably 6 hours or more, more preferably 12 hours or more, more preferably 18 hours or more, still preferably one day or more, and further more preferably one day to seven days. Cultivating the solanaceous plant for 1 hour or more with the root thereof being soaked in the treatment solution ensures sufficient chance for the salt tolerance imparting agent in the treatment solution to contact the root of the solanaceous plant, whereby the salt tolerance can be imparted more easily.

In the initial growth step, when the seedling is to be grown in a state of being supported by the supporting carrier retained within the cultivation pot, the cultivation pot may be installed in a treatment tank containing the treatment solution such that the root growing from below the supporting carrier contacts the treatment solution, thereby carrying out the salt tolerance imparting treatment. For example, the root can be brought into contact with the treatment solution by using a float which has one or more through-holes for fitting the cultivation pot thereinto and is floated on the surface of the treatment solution, and fitting the cultivation pot into the float. The cultivation pot may be detachably fitted into the through-hole of the float, or may be fixed undetachably to the through-hole of the float. Alternatively, the float and the cultivation pot may be integrally formed. As a material of the float to be floated on the surface of the treatment solution, the same material as the float to be floated on the surface of the cultivation solution described later can be used.

The larger the amount of the treatment solution used in the salt tolerance imparting treatment, the larger the amount of the salt tolerance imparting agent needed. Therefore, by reducing the amount of the treatment solution to an amount that is necessary and sufficient to allow the root of the solanaceous plant grown from the bottom surface of the cultivation pot to contact the treatment solution, the amount of the salt tolerance imparting agent required for performing the salt tolerance imparting treatment once can be suppressed. However, when the amount of the treatment solution is too small, it may become impossible to allow a sufficient amount of the salt tolerance imparting agent to contact the root of the solanaceous plant. Therefore, when a plate into which one cultivation pot is fitted is installed per treatment tank, the amount of the treatment solution contained in the treatment tank is preferably at least 5 mL.

Thereafter, the seedling to which the salt tolerance is imparted by the salt tolerance imparting step is hydroponically cultivated with the cultivation solution having a sodium chloride concentration of 1% by mass or more.

In the present invention, the sodium chloride concentration of the cultivation solution in the cultivation step is not limited as long as the sodium chloride concentration is 1% by mass or more, and may be appropriately adjusted in accordance with the salt tolerance of the solanaceous plant to be cultivated. The sodium chloride concentration of the cultivation solution is preferably 1 to 4% by mass, more preferably 1.5 to 3.8% by mass, further preferably 2 to 3.5% by mass, and particularly preferably 2.5 to 3.3% by mass.

The cultivation solution used in the present invention preferably contains magnesium chloride in addition to sodium chloride, where the amount of magnesium chloride contained is preferably 0.5% by mass or less, more preferably 0.1 to 0.5% by mass.

In addition to sodium chloride and magnesium chloride, it is preferable that the cultivation solution used in the present invention contains various nutrient components which are necessary for the growth of the solanaceous plant. The nutrient components can be appropriately adjusted according to the kind of the solanaceous plant to be cultivated. Especially, it is preferable that the cultivation solution contains elements necessary for growth of solanaceous plants in the form of salts. Examples of such elements include nitrogen, phosphorus, potassium, calcium, magnesium, sulfur, iron, manganese, copper, molybdenum, and boron. The cultivation solution may further contain elements such as aluminum and silicon in the form of salts thereof, depending on the kind of the solanaceous plant. Further, the composition of the cultivation solution may be varied according to the growth stage of the solanaceous plant.

The cultivation solution to be used in the present invention may be, for example, a solution prepared by supplementing deficient salt such as sodium chloride to commercially available liquid fertilizer or a solution obtained by diluting commercially available concentrated liquid fertilizer with sea water instead of water. Further, the cultivation solution may also be a solution obtained by appropriately adding a deficient salt such as salt of phosphorus to seawater.

In the present invention, the hydroponic cultivation in the cultivation step may be performed by a general hydroponic cultivation method, except that the sodium chloride concentration of the cultivation solution is set to 1% by mass or more. The cultivation step may be performed by a deep flow technique in which a relatively large amount of the cultivation solution is stored in the cultivation tank, or a nutrient film technique in which a culture solution is allowed to flow down little by little on a flat surface having a gentle slope.

In the deep flow technique, the replacement of the cultivation solution in the cultivation tank may be carried out by a circulation method in which the cultivation solution used is circulated, or a non-circulation method in which the cultivation solution used for a certain period of time in the cultivation tank is drained. In case of the circulation method, the cultivation solution prepared in a cultivation solution preparation tank is charged into the cultivation tank by a pump or the like, and is collected back to the cultivation solution preparation tank from the cultivation tank, and the nutrient component or the like is prepared.

For example, the deep flow technique can be carried out by using a hydroponic cultivation apparatus having: a cultivation tank for storing the cultivation solution; a cultivation pot for accommodating the solanaceous plant; and a float which has one or more through-holes for fitting the cultivation pot, and is to be floated on the surface of the cultivation solution. The cultivation pot may be detachably fitted into the through-hole of the float, or may be fixed undetachably to the through-hole of the float. Alternatively, the float and the cultivation pot may be integrally formed. The cultivation tank may be installed indoors, or may be installed outdoors.

In the case of a circulation type hydroponic cultivation apparatus, the cultivation tank includes a water supply hole for injecting the cultivation solution, and a drainage hole for draining the cultivation solution. In the case of a non-circulation type hydroponic cultivation apparatus, the cultivation tank may include both of the water supply hole and the drainage hole, or may include a water supply/drainage hole used for both of the water supply and the drainage. The water supply and drainage of the cultivation solution to and from the cultivation tank are controlled by a pump and a valve.

The cultivation pot is a container that has openings at least on its upper side and lower side, and is capable of retaining the supporting carrier. In general, the cultivation pot formed of a resin material such as polyethylene, polypropylene, or polyvinylidene chloride is used. As the supporting carrier to be retained in the cultivation pot, any of those described above can be used.

The float is formed of a material that floats on the surface of the cultivation solution in a state where the cultivation pot being used for cultivating the solanaceous plant is fitted into the through-hole. As such a material, for example, a foamed resin such as polystyrene foam or polypropylene foam is used. By fitting the cultivation pot into the float, the cultivation pot can be always positioned on the surface of the cultivation solution, regardless of a large or small amount of the cultivation solution, and even if the amount of the cultivation solution is small, the root of the solanaceous plant can be allowed to contact constantly with the cultivation solution.

The float to be floated in the cultivation tank may be one sheet, or two or more sheets. When the cultivation tank is installed outdoors, it is preferable that the float is installed to cover most of the surface of the cultivation solution, in order to prevent evaporation of the cultivation solution from the surface thereof.

In the deep flow technique, it is preferable that the hydroponic cultivation apparatus used includes oxygen supply means for keeping the dissolved oxygen content of the cultivation solution at a predetermined level or higher. As the oxygen supply means, for example, an air pump or an air sucker can be used. By installing the air pump in the cultivation tank, air including oxygen can be directly supplied to the cultivation solution in the cultivation tank. When the air sucker is used, the cultivation solution can be charged into the cultivation tank after the cultivation solution is mixed with air by passing the cultivation solution through the air sucker or the like in advance.

The pH suitable for the hydroponic cultivation varies depending on the kind of the plant, and is generally approximately 5.5 to 6.5, but the pH of the cultivation solution tends to increase as the cultivation period extends longer. Therefore, in order to stably perform the hydroponic cultivation for a long period of time, it is preferable that the hydroponic cultivation apparatus used includes a pH control means for measuring the pH of the cultivation solution periodically, and dosing an acid material in order to adjust the pH within a predetermined range as necessary. As the acid material used for the pH adjustment, for example, hydrochloric acid, sulfuric acid, or nitric acid can be used.

In the solanaceous plant production method of the present invention, the salt tolerance imparting step and the cultivation step may be performed in the same cultivation tank, or the salt tolerance imparting step may be performed in the treatment tank storing the treatment solution, followed by transferring the seedling after the treatment to the cultivation tank storing the cultivation solution.

In the initial growth step, when the salt tolerance imparting treatment is performed in the treatment tank after the seedling is grown in a state of being supported by the supporting carrier retained within the cultivation pot, the cultivation pot with the seedling may be detached from the float of the treatment tank, and may be fitted into the through-hole of the float floating on the surface of the cultivation solution stored in the cultivation tank, or the float into which the cultivation pot is embedded may be transferred from the treatment tank to be floated on the surface of the cultivation solution in the cultivation tank. The transfer means for transferring the cultivation pot or the float to the cultivation tank from the treatment tank is not particularly limited, and for example, the transfer may be performed by means of a water current, or a conveyor. When multiple cultivation pots are installed per treatment tank, it is preferable that a bubbling treatment is performed by an air pump, in order to prevent stagnation of the treatment solution, and oxygen deficiency.

When the salt tolerance imparting treatment is performed in the cultivation tank, first, the treatment solution is charged to the cultivation tank, and the root grown downward in the cultivation pot fitted into the float is brought into contact with the treatment solution to thereby perform the salt tolerance imparting treatment. In order to prevent the occurrence of concentration gradient of the salt tolerance imparting agent, it is preferable that the treatment solution is contacted with the root of the solanaceous plant while suppressing the amount of the water supply and drainage or without the water supply and drainage.

However, when the amount of the water supply and drainage is small or the water supply and drainage is not performed, the stagnation may occur in the cultivation tank, causing adverse effect on the solanaceous plant itself. Therefore, it is preferable that the treatment solution is suitably stirred by the bubbling treatment with an air pump.

After the salt tolerance imparting treatment, the treatment solution in the cultivation tank is drained, and subsequently, the cultivation solution prepared in advance in another tank is supplied to the cultivation tank. Then, the cultivation step is initiated by supplying water and draining under normal conditions. When the salt tolerance imparting agent is composed of a material, such as the microorganism, which does not adversely affect the solanaceous plant even in the event of excessive intake of the material, the cultivation solution may be supplied without draining the treatment solution, and the water supply and drainage may be initiated under the normal water supply and drainage conditions.

The cultivation with the cultivation solution having a sodium chloride concentration of 1% by mass or more (hereinafter, also referred to as “under the high salinity environment”) need not necessarily be performed throughout the entire cultivation period after the salt tolerance imparting treatment. For example, the cultivation under the high salinity environment may be performed only for an arbitrary period after the salt tolerance imparting treatment. In this case, it is preferable that the cultivation under the high salinity environment is performed for a predetermined period immediately after the salt tolerance imparting treatment. It is speculated that, by performing the cultivation under the high salinity environment at an arbitrary time immediately after the salt tolerance imparting treatment, the salt tolerance imparted by the salt tolerance imparting treatment is maintained, whereby the shelf life of the plant improves. The period of the cultivation under the high salinity environment (i.e., the number of days for performing cultivation using the cultivation solution) is not particularly limited, but for example, may be a period of approximately ⅓ of the entire cultivation period, a period of approximately ½ of the entire cultivation period, or a period of approximately ⅔ of the entire cultivation period, from immediately after the salt tolerance imparting treatment. More specifically, the lower limit of the number of days for the cultivation is preferably ⅓ or more, more preferably ½ or more, still more preferably ⅔ or more, still more preferably 9/13 or more, and even more preferably 10/13 or more, relative to the entire cultivation period. From the viewpoint of obtaining the effects of the present invention, it is preferable that the cultivation under the high salinity environment is performed throughout the entire cultivation period, from immediately after the salt tolerance imparting treatment. In order to impart salt tolerance to the plant at an early stage, the cultivation medium may be switched to the cultivation solution containing sodium chloride immediately after the salt tolerance imparting agent is brought into contact with the plant, or from a point of time when the plant is imparted with salt tolerance even if only slightly.

The cultivation using the cultivation solution may be terminated before harvesting, and the cultivation may be switched to a cultivation that does not use the cultivating solution, or uses a solution devoid of some of the components of the cultivation solution (for example, a solution lacking sodium chloride) or a dilution of the cultivation solution (for example, a nutrient solution having a lower sodium chloride concentration than the cultivating solution), but it is most preferable to perform the cultivation until harvesting.

The upper limit of the number of days for performing the cultivation using the cultivation solution is not particularly limited, and may be preferably ⅞ or less, more preferably 9/10 or less, further preferably 12/13 or less, relative to the entire cultivation period. However, the upper limit is most preferably the entire cultivation period (1/1). That is, the range of the number of days for performing the cultivation using the cultivation solution is preferably 1/3 to 1/1, more preferably 1/2 to 1/1, more preferably 2/3 to 1/1, more preferably 9/13 to 1/1, still more preferably 10/13 to 1/1, relative to the entire cultivation period.

The “entire cultivation period” means a period (number of days) of from immediately after initiation of contacting the plant with the salt tolerance imparting agent until harvesting of the fruit of the solanaceous plant.

If the seedling with insufficient salt tolerance imparted is cultivated under the high salinity environment for a certain period in the cultivation step, the seedling withers. The withered plant causes the rotting, and allows undesirable bacteria or the like to proliferate in the cultivation solution. In such a case, despite the efforts made to impart the salt tolerance to the seedling, the seedling may wither by the disease or the like, due to contamination of the cultivation solution. Therefore, it is preferable that a removal step of removing the withered seedling is performed after the salt tolerance imparting step or during the cultivation step. Especially when the salt tolerance imparting step is performed by using the treatment solution having a sodium chloride concentration of 1% by mass or more, it is preferable that the removal step is performed after the salt tolerance imparting step and before initiation of the cultivation step. In the cultivation of agricultural crops, the yield in an actual cultivation area can be improved by removing the withered seedling from the cultivation tank.

When the plant cultivation is performed for a certain period under the high salinity environment after initiation of the salt tolerance imparting treatment, the seedling which has grown without withering can be confirmed to be a solanaceous plant having the salt tolerance thereof surely improved by the salt tolerance imparting agent. By removing the withered seedling, the salt tolerant seedling produced according to the present invention can acquire quality assurance as a salt tolerant seedling.

The cultivation step may be performed indoors using the cultivation tank installed indoors, or may be performed outdoors in the open air using the cultivation tank installed outdoors.

The solanaceous plant production method of the present invention may further include a selection step of selecting, from the harvested solanaceous plant, those satisfying the above-mentioned respective ranges with respect to the amino acid contents of the edible part of the solanaceous plant.

In the solanaceous plant production method according to the present invention, when the concentration of sodium chloride in the cultivation solution is increased, the total amino acids content and the serine content can be increased. Therefore, in the solanaceous plant production method according to the present invention, the total amino acids content and serine content of the edible portion of the solanaceous plant can be controlled by adjusting the sodium chloride concentration of the cultivation solution and the period (number of days) for cultivation.

Especially, it is preferable to adjust the serine content by the method for controlling the serine content of the solanaceous plant as described above in the cultivation step. This makes it possible to easily and surely control the serine content to a desired value.

The solanaceous plant according to the present invention makes it possible to take a large amount of functional components while eating vegetables which are natural foods. The solanaceous plant according to the present invention contains many kinds of amino acids at high concentrations; therefore, the solanaceous plant is expected to exhibit multiple functions and higher activity with respect to each function, and is expected to enhance such activity via multiple mechanisms. For example, the solanaceous plant according to the present invention contain serine at a high concentration, which has sleep-improving action. Similarly, when the solanaceous plant contains glycine at a high concentration, which also has sleep-improving action, enhanced sleep-improving action via multiple mechanisms can be expected. Regarding glycine, it has been suggested that glycine acts on NMDA receptor (NMDAR) as an excitatory neurotransmission regulator to thereby improves sleep. Regarding serine, it has been suggested that sleep-improving action of serine is via a mechanism mediated by the GABAA receptor. That is, even with the same effect of improving sleep, synergistic effects can be expected through multiple mechanisms by containing multiple components.

Various amino acids contained in the solanaceous plant exhibit high effects as their amounts and concentrations increase. As the amounts and concentrations of the amino acids contained in the solanaceous plant increase, large amounts of the amino acids can be ingested with less amount of the plant, whereby the solanaceous plant of the present invention can reduce the number of the solanaceous plant that should be ingested to take desired amounts of amino acids.

The solanaceous plant according to the present invention are enriched in functional components. In some cases, genetically modified vegetables are sometimes not favored due to domestic regulations of each country, impression given by recombinant vegetables, etc. On the other hand, according to the configuration of the present invention, it is possible to provide solanaceous plants enriched in functional components even without using gene modification technology.

The present invention can provide solanaceous plants that have excellent flavor as well as the functionality. For example, the solanaceous plants may be enriched in glutamic acid or the like known as a flavor component in addition to the functional components, whereby various functional components can be deliciously ingested.

### <<Method for Identifying Solanaceous Plant Cultivation Method>>

The method of the present invention for identifying a cultivation method includes: measuring amino acid contents in terms of amounts (mg) of respective amino acids present per 100 g of an edible portion of a solanaceous plant (amino acid contents measuring step);

calculating the total amino acid content α (mg) per 100 g of the edible portion of the solanaceous plant from the amino acid contents (total amino acid content calculating step), calculating a serine content (3 (mol %) relative to 100 mol % of total amino acids (serine content calculating step), and determining that the solanaceous plant is one produced by hydroponics with a cultivation solution containing 1% by mass or more of sodium chloride when the total amino acid content α is not less than a predetermined value α0 and the serine content β is not less than a predetermined value (3S (cultivation method identifying step).

### <Amino Acid Contents Measuring Step>

In the amino acid contents measuring step, the amino acid contents in terms of amounts (mg) of respective amino acids present per 100 g of an edible portion of a solanaceous plant are calculated.

The subject amino acids and the method for measuring the amounts of amino acids are as described above.

Further, the degree of ripening of tomato and the types of solanaceous plants are also as described above.

### <Total Amino Acid Content Calculating Step>

In the total amino acid content calculating step, the total amino acid content is calculated by adding up the amino acid contents measured in the amino acid contents measuring step.

Further, the amino acid contents are converted into molar amounts and added up to calculate the total amino acid content (mol).

In the present specification, the total amino acids content are as described above.

### <Serine Content Calculating Step>

In the serine content calculating step, a serine content (mol %) is calculated with the proviso that the total amino acid content calculated in the total amino acid content calculating step is 100 mol %.

### <Cultivation Method Identifying Step>

In the cultivation method identifying step, the solanaceous plant is one produced by hydroponics with a cultivation solution containing 1% by mass or more of sodium chloride when the total amino acids content α calculated in the <total amino acid content calculating step> is not less than a predetermined value α₀ and the serine content β calculated in the <serine content calculating step> is not less than a predetermined value β_{S}.

The predetermined value α₀ is preferably 300 mg, and more preferably 400 mg.

The predetermined value β_{S} is preferably 8 mol %, and more preferably 8.5 mol %.

The method of the present invention for identifying solanaceous plant cultivation method may further includes: calculating a glutamate content (mol %) relative to 100 mol % of total amino acids content (glutamate content calculating step), and checking whether the glutamate content is not less than a predetermined value PE (glutamate content checking step). When the method of the present invention includes the glutamate content checking step, the solanaceous plant cultivation method can be more accurately identified.

The method of the present invention for identifying solanaceous plant cultivation method may further includes: calculating a glutamate content (mol %) relative to 100 mol % of total amino acids content (aspartate content calculating step), and checking whether the aspartate content is not less than a predetermined value β_{D} (aspartate content checking step). When the method of the present invention includes the aspartate content checking step, the solanaceous plant cultivation method can be more accurately identified.

The method of the present invention for identifying tomato cultivation method may further includes: calculating an arginine content (mol %) relative to 100 mol % of total amino acids content (arginine content calculating step), and checking whether the arginine content is not less than a predetermined value PR (arginine content checking step). When the method of the present invention includes the arginine content checking step, the solanaceous plant cultivation method can be more accurately identified.

The method of the present invention for identifying tomato cultivation method may further includes: calculating an isoleucine content (mol %) relative to 100 mol % of total amino acids content (isoleucine content calculating step), and checking whether the isoleucine content is not less than a predetermined value β_{T} (isoleucine content checking step). When the method of the present invention includes the isoleucine content checking step, the solanaceous plant cultivation method can be more accurately identified.

The method of the present invention for identifying solanaceous plant cultivation method may further includes: calculating an alanine content (mol %) relative to 100 mol % of total amino acids content (alanine content calculating step), and checking whether the alanine content is not less than a predetermined value β_{A} (alanine content checking step). When the method of the present invention includes the alanine content checking step, the solanaceous plant cultivation method can be more accurately identified.

The method of the present invention for identifying solanaceous plant cultivation method may further includes: calculating a lysine content (mol %) relative to 100 mol % of total amino acids content (lysine content calculating step), and checking whether the lysine content is not less than a predetermined value β_{K} (lysine content checking step). When the method of the present invention includes the lysine content checking step, the solanaceous plant cultivation method can be more accurately identified.

The method of the present invention for identifying solanaceous plant cultivation method may further includes: calculating a histidine content (mol %) relative to 100 mol % of total amino acids content (histidine content calculating step), and checking whether the histidine content is not less than a predetermined value β_{II} (histidine content checking step). When the method of the present invention includes the histidine content checking step, the solanaceous plant cultivation method can be more accurately identified.

The method of the present invention for identifying tomato cultivation method may further includes: calculating a phenylalanine content (mol %) relative to 100 mol % of total amino acids content (phenylalanine content calculating step), and checking whether the phenylalanine content is not less than a predetermined value β_{F} (phenylalanine content checking step). When the method of the present invention includes the phenylalanine content checking step, the solanaceous plant cultivation method can be more accurately identified.

The method of the present invention for identifying tomato cultivation method may further includes: calculating a tyrosine content (mol %) relative to 100 mol % of total amino acids content (tyrosine content calculating step), and checking whether the tyrosine content is not less than a predetermined value β_{Y} (tyrosine content checking step). When the method of the present invention includes the tyrosine content checking step, the solanaceous plant cultivation method can be more accurately identified.

The method of the present invention for identifying solanaceous plant cultivation method may further includes: calculating a leucine content (mol %) relative to 100 mol % of total amino acids content (leucine content calculating step), and checking whether the leucine content is not less than a predetermined value β_{L} (leucine content checking step). When the method of the present invention includes the leucine content checking step, the solanaceous plant cultivation method can be more accurately identified.

The method of the present invention for identifying solanaceous plant cultivation method may further includes: calculating a methionine content (mol %) relative to 100 mol % of total amino acids content (methionine content calculating step), and checking whether the methionine content is not less than a predetermined value β_{M} (methionine content checking step). When the method of the present invention includes the methionine content checking step, the solanaceous plant cultivation method can be more accurately identified.

The method of the present invention for identifying solanaceous plant cultivation method may further includes: calculating a valine content (mol %) relative to 100 mol % of total amino acids content (valine content calculating step), and checking whether the valine content is not less than a predetermined value β_{L} (valine content checking step). When the method of the present invention includes the valine content checking step, the solanaceous plant cultivation method can be more accurately identified.

The method of the present invention for identifying solanaceous plant cultivation method may further includes: calculating a glycine content (mol %) relative to 100 mol % of total amino acids content (glycine content calculating step), and checking whether the glycine content is not less than a predetermined value 13L (glycine content checking step). When the method of the present invention includes the glycine content checking step, the solanaceous plant cultivation method can be more accurately identified.

The method of the present invention for identifying tomato cultivation method may further includes: calculating a proline content (mol %) relative to 100 mol % of total amino acids content (proline content calculating step), and checking whether the proline content is not less than a predetermined value 13p (proline content checking step). When the method of the present invention includes the proline content checking step, the solanaceous plant cultivation method can be more accurately identified.

The method of the present invention for identifying solanaceous plant cultivation method may further includes: calculating a threonine content (mol %) relative to 100 mol % of total amino acids content (threonine content calculating step), and checking whether the threonine content is not less than a predetermined value β_{T} (threonine content checking step). When the method of the present invention includes the threonine content checking step, the solanaceous plant cultivation method can be more accurately identified.

The method of the present invention for identifying solanaceous plant cultivation method may further includes: calculating a tryptophan content (mol %) relative to 100 mol % of total amino acids content (tryptophan content calculating step), and checking whether the tryptophan content is not less than a predetermined value β_{W} (tryptophan content checking step). When the method of the present invention includes the tryptophan content checking step, the solanaceous plant cultivation method can be more accurately identified.

### <<Solanaceous Plant Cultivation Method>>

As a solanaceous plant cultivation method to be identified by the method of the present invention, there can be mentioned the same method as the above-mentioned

### <<Solanaceous Plant Production Method>>.

### <<Estimation Method for Exposure to Sodium Chloride>>

From the total amino acids content α and the serine content (3, the exposure of solanaceous plant to sodium chloride can be estimated.

First, solanaceous plants are cultivated under multiple different cultivation conditions in which the concentration of sodium chloride in the cultivation solution is varied.

From the concentration of sodium chloride in the cultivation solution and the number of days for cultivation in the cultivation solution, the exposure to sodium chloride is calculated by the following formula (1).

Exposure to sodium chloride=[concentration of sodium chloride in cultivation solution (% by mass)]×[days of cultivation using cultivation solution(days)] (1)

Subsequently, the amino acid contents in terms of amounts of respective amino acids present per 100 g of an edible portion of the solanaceous plant cultivated under the cultivation condition described above are measured to calculate the total amino acid content (mg).

The values of the total amino acid content and the exposure to sodium chloride are respectively plotted on the horizontal axis and the vertical axis to create a calibration curve (1).

Further, the serine molar content (mol %) per 100 mol % of total amino acids content is calculated.

The values of the serine content and the exposure to sodium chloride are respectively plotted on the horizontal axis and the vertical axis to create a calibration curve (2).

With respect to a solanaceous plant produced by unknown cultivation method, the values of the total amino acids content α and the serine content (3 are determined, and the exposure of tomato to sodium chloride is estimated from the calibration curves (1) and (2).

### EXAMPLES

Hereinbelow, the present invention will be described with reference to Examples which, however, should not be construed as limiting the present invention.

### Example 1

After a surface of a seed of a tomato (Kanpuku) was sterilized with hypochlorous acid, the seed was sowed on a sponge sufficiently including water (freshwater). After budding, a seedling was grown for two weeks, and was grown for one week in the natural environment while gradually lowering humidity in order to acclimatize the seedling to the environment. The seedling was planted in a hydroponic cultivation bed, and was further grown for several days.

Microorganisms described in Plant, Cell and Environment, (2009) 32, 1682-1694 were incubated and centrifuged, thereby obtaining a pelletized microorganisms.

A treatment solution was obtained by resuspending the pelletized microorganisms obtained above in a culture solution. Using this treatment solution, a salt tolerance imparting treatment was performed while allowing the roots of the plant to be in contact with the microorganisms for 12 hours.

Next, the treatment solution in the water tank of the hydroponic cultivation bed was filled with a cultivation solution (sodium chloride concentration of 1% by mass), and hydroponic cultivation was performed in a greenhouse for 10 weeks. In the cultivation solution used in this Example, various nutrients necessary for the hydroponic cultivation were added.

### Example 2

A tomato was cultivated in the same manner as in Example 1 except that the concentration of sodium chloride in the salt water was changed to 2% by mass.

### Example 3

A tomato was cultivated in the same manner as in Example 1 except that the concentration of sodium chloride in the salt water was changed to 3% by mass.

### Comparative Example 1

A tomato was cultivated in the same manner as in Example 1 except that freshwater was used instead of the salt water.

Tomato fruits were harvested from the tomato plants cultivated in Examples 1 to 3 and Comparative Example 1, and the amounts of amino acids were measured with respect to the edible portion of each of the tomato fruits. The results are shown in Table 1. In Table 1, the figures with “mg/100 g” denote the contents of amino acids per 100 g of an edible portion. The figures with “mol” denote the number of moles of amino acids. The figures with “mol %” denote the concentrations (mol %) of amino acids per 100 mol % of total amino acids content.

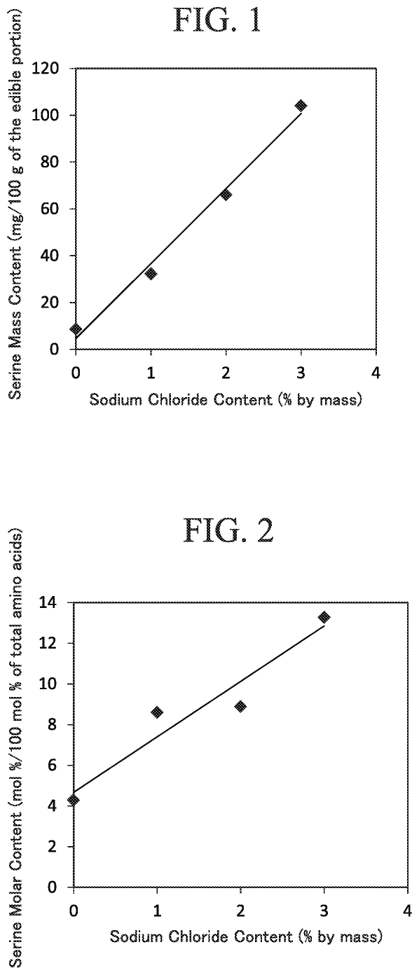

Further, FIG. 1 is a graph showing the relationship between sodium chloride content and serine content (mg/100 g of edible portion). FIG. 2 is a graph showing the relationship between sodium chloride content and serine content (mol %/100 mol % of total amino acids content).

With respect to each of the tomato fruits harvested from the tomato plants cultivated in Examples 1 to 3 and Comparative Example 1, the amount of sodium contained in the edible portion thereof was measured. In each of the Examples and Comparative Example, the amount of sodium was measured with respect to the fruits of a plurality of tomatoes, and the average value was calculated. The amount (average value) of sodium was 0.24% by mass in Example 1, 0.39% by mass in Example 2, and 0.073% by mass in Comparative Example 1, each based on the total mass of edible portion of the tomato fruit.

### <Evaluation>

Each of the tomatoes obtained in Examples and Comparative Examples was cut into quarters, and was eaten by a panel of 6 people who were not informed of the cultivation conditions of the tomatoes. All of the 6 people were able to distinguish the tomato of Comparative Example 1 from the 4 types in total of the tomatoes including those of Examples 1 to 3 and Comparative Example 1. For all of the 6 people, the tomatoes of Examples 1 to 3 cultivated at sodium chloride concentrations of 1% by mass, 2% by mass and 3% by mass gave different impression from the tomato of Comparative Example 1. Especially, the tomato cultivated at a sodium chloride concentration of 3% by mass gave an impression that the flavor is clear.

TABLE 1

| | Comparative Example 1 | | | Example 1 | | | Example 2 | | | Example 3 | | |
|---|---|---|---|---|---|---|---|---|---|---|---|---|
| | mg/ | | | mg/ | | | mg/ | | | mg/ | | |
| Amino Acid | 100 g | mol | mol % | 100 g | mol | mol % | 100 g | mol | mol % | 100 g | mol | mol % |
| Arginine | 5.9 | 3.39 | 1.78 | 4.0 | 2.30 | 0.64 | 6.3 | 3.62 | 0.51 | 23 | 13.20 | 1.77 |
| Lysine | 3.6 | 2.46 | 1.29 | 4.4 | 3.01 | 0.84 | 7.0 | 4.79 | 0.68 | 21 | 14.36 | 1.93 |
| Histidine | 4.6 | 2.96 | 1.56 | 7.6 | 4.90 | 1.37 | 12.1 | 7.80 | 1.10 | 26 | 16.76 | 2.25 |
| Phenylalanine | 5.9 | 3.57 | 1.87 | 6.4 | 3.87 | 1.08 | 5.9 | 3.57 | 0.51 | 27 | 16.34 | 2.19 |
| Tyrosine | 1.8 | 0.99 | 0.52 | 1.7 | 0.94 | 0.26 | 1.6 | 0.88 | 0.13 | 10 | 5.52 | 0.74 |
| Leucine | 3.2 | 2.44 | 1.28 | 3.4 | 2.59 | 0.72 | 4.1 | 3.13 | 0.44 | 8 | 6.10 | 0.82 |
| Isoleucine | 2.9 | 2.21 | 1.16 | 6.8 | 5.18 | 1.45 | 8.3 | 6.33 | 0.90 | 18 | 13.72 | 1.84 |
| Methionine | 1.2 | 0.84 | 0.44 | 1.0 | 0.70 | 0.20 | 1.3 | 0.91 | 0.13 | 4 | 2.81 | 0.38 |
| Valine | 2.8 | 2.39 | 1.25 | 4.1 | 3.50 | 0.98 | 3.8 | 3.24 | 0.46 | 8 | 6.83 | 0.92 |
| Alanine | 3.6 | 0.74 | 0.39 | 9.1 | 1.86 | 0.52 | 22.9 | 4.68 | 0.66 | 55 | 11.25 | 1.51 |
| Glycine | 1.1 | 1.47 | 0.77 | 2.0 | 2.66 | 0.74 | 2.9 | 3.86 | 0.55 | 9 | 11.99 | 1.61 |
| Proline | 1.2 | 1.04 | 0.55 | 33.9 | 29.44 | 8.23 | 88.3 | 76.70 | 10.86 | 26 | 22.58 | 3.03 |
| Glutamic Acid | 185.9 | 126.35 | 66.30 | 321.9 | 218.79 | 61.15 | 650.1 | 441.85 | 62.56 | 573 | 389.45 | 52.21 |
| Serine | 8.6 | 8.18 | 4.29 | 32.3 | 30.74 | 8.59 | 66.0 | 62.80 | 8.89 | 104 | 98.96 | 13.27 |
| Threonine | 5.4 | 4.53 | 2.38 | 12.3 | 10.33 | 2.89 | 18.0 | 15.11 | 2.14 | 26 | 21.83 | 2.93 |
| Aspartic Acid | 34.6 | 26.19 | 13.74 | 47.9 | 36.25 | 10.13 | 87.3 | 66.08 | 9.35 | 122 | 92.34 | 12.38 |
| Tryptophan | 1.6 | 0.78 | 0.41 | 1.3 | 0.64 | 0.18 | 1.5 | 0.73 | 0.10 | 4 | 1.96 | 0.26 |
| Cystine | 0.0 | 0.03 | 0.02 | 0.1 | 0.08 | 0.02 | 0.3 | 0.25 | 0.04 | 0 | 0.00 | 0.00 |
| Total | 268.0 | 190.5786 | 100.00 | 496.1 | 357.7863 | 100.00 | 981.1 | 706.3361 | 100.00 | 1064 | 746.0064 | 100.00 |

The tomato fruits obtained in Examples 1 to 3 were found to have higher total amino acid content than that of the tomato fruit obtained in Comparative Example 1. In addition, the tomato fruits obtained in Examples 1 to 3 were found to have higher serine contents than that of the tomato fruits obtained in Comparative Example 1.

The results of Examples 1 to 4 revealed that the serine content increases as the sodium chloride concentration of the cultivation solution increases. An almost proportional relationship was observed between the sodium chloride concentration of the cultivation solution and the serine content. Further, a proportional relationship was also observed between the sodium chloride concentration of the cultivation solution and the serine content (mol %) relative to the total amino acid content of the tomato. Thus, it has been found that the serine content can be controlled by adjusting the sodium chloride concentration of the cultivation solution.

### (Method for Identifying Tomato Cultivation Method)

Tomato fruits were harvested from the tomato plants cultivated in Examples 1 to 3 and Comparative Example 1, and the amino acid contents were measured with respect to the edible portion of each of the tomato fruits. The amino acid contents were added up to calculate the total amino acid content.

Further, the amino acid contents were converted into molar amounts and added up to calculate the serine content (mol %).

In all of Examples 1 to 3, the total amino acid content with respect to the edible portion of the harvested tomato fruit was not less than 300 mg. Further, in all of Examples 1 to 3, the serine content was not less than 8 mol %.

In Comparative Example 1, the total amino acid content with respect to the edible portion of the harvested tomato fruit was less than 300 mg. Further, in Comparative Example 1, the serine content was less than 8 mol %.

Thus, in Examples 1 to 3, the total amino acid content was not less than the predetermined value (300 mg in this instance) and the serine content was not less than the predetermined value (8 mol % in this instance). From this, it was determined that the tomatoes cultivated in Examples 1 to 3 were those cultivated by hydroponic cultivation with a cultivation solution containing 1% by mass or more of sodium chloride.

### INDUSTRIAL APPLICABILITY

The present invention can provide a solanaceous plant with higher concentrations of amino acids as functional components, as compared to conventional solanaceous plants. Further, the present invention can provide a solanaceous plant with higher serine content among all amino acids, as compared to conventional solanaceous plants. Thus, in particular, the present invention can provide a solanaceous plant which enables efficient ingestion of serine. The present invention can also provide a method for identifying solanaceous plant cultivation method. The present invention can provide a method for controlling a serine content of a solanaceous plant.

1. A method for controlling a serine content of a solanaceous plant, comprising performing cultivation of a solanaceous plant using a cultivation solution containing sodium chloride, while adjusting one or both of concentration of sodium chloride in the cultivation solution and number of days for performing the cultivation using the cultivation solution.

2. The method according to claim 1, wherein the cultivation is hydroponic cultivation.

3. The method according to claim 1, wherein the concentration of sodium chloride in the cultivation solution is 1% by mass or more.

4. The method according to claim 1, wherein the number of days for performing the cultivation using the cultivation solution is ½ or more of the entire cultivation period.

5. The method according to claim 1, wherein the solanaceous plant is a tomato.

6. A solanaceous plant having a serine molar content of 8 mol % or more per 100 mol % of total amino acid content of the solanaceous plant.

7. The solanaceous plant according to claim 6, which has a total amino acid content of 300 mg or more per 100 g of an edible portion of the solanaceous plant.

8. The solanaceous plant according to claim 5, which has a serine mass content of 30 mg or more per 100 g of the edible portion.

9. The solanaceous plant according to claim 6, which is a tomato.

10. A cultivation method identifying method for degerming whether a solanaceous plant is one produced by hydroponics with a cultivation solution containing 1% by mass or more of sodium chloride, comprising:
measuring amino acid contents in terms of amounts (mg) of respective amino acids present per 100 g of an edible portion of a solanaceous plant,
calculating a total amino acids content α (mg) per 100 g of the edible portion of the solanaceous plant from the amino acid contents,
calculating a serine content β (mol %) relative to 100 mol % of total amino acids content, and
determining that the solanaceous plant is one produced by hydroponic cultivation with a cultivation solution containing 1% by mass or more of sodium chloride when the total amino acids content α is not less than a predetermined value α₀ and the serine content β is not less than a predetermined value β_{S}.

11. The method according to claim 8, wherein the predetermined value α₀ is 300 mg, and
the predetermined value β_{S} is 8 mol %.

12. The method according to claim 10, wherein the solanaceous plant is a tomato.


SEQUENCE LISTING (1 sequence)

SEQ ID NO: 1 - DNA, 1399 bp, Pseudomonas corrugata
  16S rDNA (partial)
tgcagtcgagcggtagagaggtgcttgcacctcttgagagcggcggacgggtgagtaatgcctaggaatctgcctggtagtgggggataacgctcggaaacgggcgctaataccgcatacgtcctacgggagaaagcaggggaccttcgggccttgcgctatcagatgagcctaggtcggattagctagttggtgaggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcaattacctaatacgtagttgttttgacgttaccgacagaataagcaccggctaactctgtgccagcagccgcggtaatacagagggtgcaagcgttaatcggaattactgggcgtaaagcgcgcgtaggtggtttgttaagttggatgtgaaatccccgggcttaacctgggaactgcattcaaaactgacaagctagagtatggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcaactagccgttgggagccttgagctcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttccagagatggattggtgccttcgggagcattgagacaggtgctgcatggctgtcgtcagctcgtgtcgtgagatgttgggttaagtcccgtaacgagcgcaacccttgtccttagttaccagcacgttatggtgggcactctaaggagactgccggtgacaaaccggaggaaggtggggatgacgtcaagtcatcatggcccttacggcctgggctacacacgtgctacaatggtcggtacagagggttgccaagccgcgaggtggagctaatcccacaaaaccgatcgtagtccggatcgcagtctgcaactcgactgcgtgaagtcggaatcgctagtaatcgcgaatcagaatgtcgcggtgaatacgttcccgggccttgtacacaccgcccgtcacaccatgggagtgggttgcaccagaagtagctagtctaaccttcggggggac